(19) United States
(12) Patent Application Publication
(10) Pub. No.: US 2007/0172852 A1
(43) Pub. Date: Jul. 26, 2007

(54) Non-destructive Quality Control Methods for Microarrays

(75) Inventor: Michael R. Zimmerling, Chandler, AZ (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/626,053

(22) Filed: Jan. 23, 2007

Related U.S. Application Data
(60) Provisional application No. 60/761,846, filed on Jan. 24, 2006.

(51) Int. Cl. C12Q 1/68; G01N 33/00

(52) U.S. Cl. 435/6; 436/94

(57) ABSTRACT

Methods for non-destructive quality control of a synthetic round of fabrication of high density oligonucleotide arrays are disclosed.

### BACKGROUND OF THE INVENTION

Biological chips or microarrays contain large numbers of molecular probes arranged in an array format, each probe ensemble assigned a specific location. Microarrays have been produced in which each location has a scale of, for example, ten microns. The arrays can be used to determine whether target molecules interact with any of the probes on the array. After the array is exposed to target molecules under selected test conditions, scanning devices can examine each location in the array and determine whether a target molecule has interacted with the probe at that location.

Biological chips are useful in a variety of screening techniques for obtaining information about either the probes or the target molecules. For example, a library of peptides can be used as probes to screen for drugs. The peptides can be exposed to a receptor, and those probes that bind to the receptor can be identified.

Biological chips wherein the probes are oligonucleotides (“oligonucleotide arrays”) show particular promise. Arrays of nucleic acid probes can be used to extract sequence information from nucleic acid samples. The samples are exposed to the probes under conditions that allow hybridization. The arrays are then scanned to determine to which probes the sample molecules have hybridized. One can obtain sequence information by selective tiling of the probes with particular sequences on the arrays, and using algorithms to compare patterns of hybridization and non-hybridization. This method is useful for sequencing nucleic acids. It is also useful in diagnostic screening for genetic diseases or for the presence of a particular pathogen or a strain of pathogen.

The scaled-up manufacturing of oligonucleotide arrays requires application of quality control standards both for determining the quality of chips under current manufacturing conditions and for identifying optimal conditions for their manufacture, preferably non-destructive quality control methods. Quality control, of course, is not limited to manufacture of chips, but also to the conditions under which they are stored, transported, and used.

### SUMMARY OF THE INVENTION

One aspect of this invention provides non-destructive quality control (QC) methods for testing of fabrication, e.g., a synthetic round of fabrication, of a high density oligonucleotide array, wherein the synthetic round of fabrication comprises exposing a substrate in a flow cell, the substrate having a density exceeding 400 protected reactive species per cm², wherein the reactive species is protected with a photolabile protecting group and protected reactive species is selected from the group consisting of a protected terminal hydroxyl group of a linker that is bound to the substrate and directed away from the substrate, a protected 5′ hydroxyl group of a naturally or non-naturally occurring 2′-deoxyribonucleotide, and a protected 3′ hydroxyl group of a naturally or non-naturally occurring 2′-deoxyribonucleotide; selectively irradiating the substrate with light of a wavelength to remove a preselected amount of photolabile protecting groups to provide exposed reactive groups; providing an MOS block with a tube leading to the flow cell containing a first solution of a naturally or non-naturally occurring 2′-deoxyribonucleotide-3′-phosphoramidite, wherein the phosphoramidite is located at the 3′ position and the 5′ hydroxyl group is protected by a photolabile protecting group or the phosphoramidite is located at the 5′ position and the 3′ hydroxyl group is protected by a photolabile protecting group; performing an analytical measurement of the first solution to determine a stock solution concentration of phosphoramidite; flushing the flow cell with the first solution from the MOS block to couple the deoxyribonucleotides to the reactive groups, producing a waste phosphoramidite solution; testing a small volume of the waste solution to determine a waste solution concentration of phosphoramidite; comparing the stock and waste concentrations of phosphoramidites to establish a QC parameter.

### BRIEF DESCRIPTION OF THE FIGURES

FIG. 1 shows a general scheme for light-directed oligonucleotide synthesis.

### DETAILED DESCRIPTION OF THE INVENTION

This invention provides methods for optimizing the production, storage, and use of oligonucleotide arrays produced by spatially directed oligonucleotide synthesis and, in particular, light-directed oligonucleotide synthesis. As used in quality control procedures for manufacturing oligonucleotide arrays, the methods can involve manufacturing the arrays in high volume, and testing selected arrays for various quality parameters such as nucleotide coupling efficiency; amount of deprotection of oligonucleotides; oligonucleotide integrity, e.g., amount of depurination; or amount of double stranded oligonucleotides in the array. Manufacturing arrays in high volume means manufacturing at least 10, 50, 500, 1000, 2000, 5000 or 10,000 oligonucleotide arrays per day from a single fabricating machine or in a single fabrication facility.

As used herein, “spatially directed oligonucleotide synthesis” refers to any method of directing the synthesis of an oligonucleotide to a specific location on a substrate. Methods for spatially directed oligonucleotide synthesis include, without limitation, light-directed oligonucleotide synthesis, microlithography, application by ink jet, microchannel deposition to specific locations and sequestration with physical barriers. In general these methods involve generating active sites, usually by removing protective groups; and coupling to the active site a nucleotide which, itself, optionally has a protected active site if further nucleotide coupling is desired.

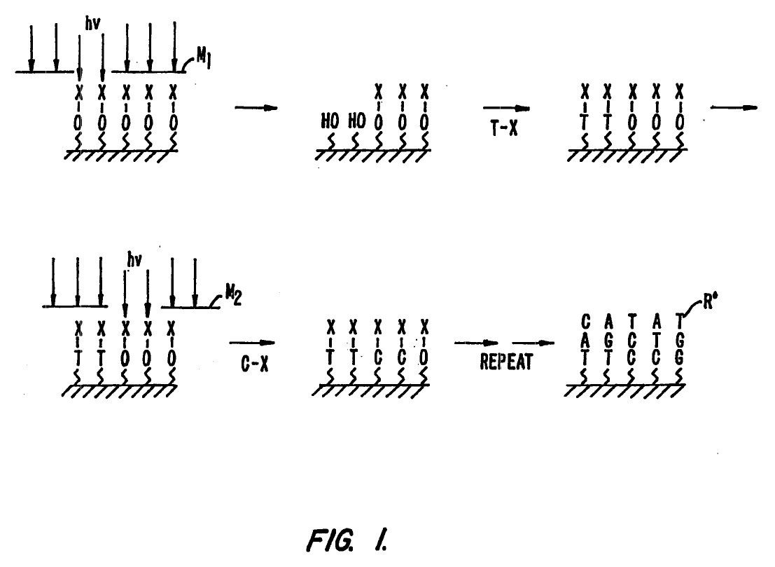

In one embodiment oligonucleotide arrays are synthesized at specific locations by light-directed oligonucleotide synthesis. The pioneering techniques of this method are disclosed in U.S. Pat. No. 5,143,854; PCT WO 92/10092; PCT WO 90/15070; U.S. Pat. No. 5,571,639 and U.S. patent application Ser. Nos. and Ser. Nos. 07/624,120, and 08/082,937, incorporated herein by reference in their entirety for all purposes. The basic strategy of this process is outlined in FIG. 1. The surface of a solid support modified with linkers and photolabile protecting groups (—O—X) is illuminated (hv) through a photolithographic mask (M1), yielding reactive hydroxyl groups (HO) in the illuminated regions. A 3′-O-phosphoramidite-activated deoxynucleoside (protected at the 5′-hydroxyl with a photolabile group, TX) is then presented to the surface and coupling occurs at sites that were exposed to light. Following the optional capping of unreacted active sites and oxidation, the substrate is rinsed and the surface is illuminated (hv) through a second mask (M2), to expose additional hydroxyl groups for coupling to the linker. A second 5′-protected, 3′-O-phosphoramidite-activated deoxynucleoside (CX) is presented to the surface. The selective photodeprotection and coupling cycles are repeated until the desired set of products is obtained. Photolabile groups are then optionally removed and the sequence is, thereafter, optionally capped. Side chain protective groups, if present, are also removed. Since photolithography is used, the process can be miniaturized to generate high-density arrays of oligonucleotide probes. Furthermore, the sequence of the oligonucleotides at each site is known.

This general process can be modified. For example, the nucleotides can be natural nucleotides, chemically modified nucleotides or nucleotide analogs, as long as they have activated hydroxyl groups compatible with the linking chemistry. The protective groups can, themselves, be photolabile. Alternatively, the protective groups can be labile under certain chemical conditions, e.g., acid labile. In this example, the surface of the solid support can contain a composition that generates acids upon exposure to light. Thus, exposure of a region of the substrate to light generates acids in that region that remove the protective groups in the exposed region. Also, the synthesis method can use 3′-protected 5′-O-phosphoramidite-activated deoxynucleoside. In this case, the oligonucleotide is synthesized in the 5′ to 3′ direction, which results in a free 5′ end.

The general process of removing protective groups by exposure to light, coupling nucleotides (optionally competent for further coupling) to the exposed active sites, and optionally capping unreacted sites is referred to herein as “light-directed nucleotide coupling.”

Methods of spatially directed synthesis can be used for creating arrays of other kinds of molecules as well, and these arrays also can be tested by the methods of this invention. For example, using the strategies described above, spatially patterned arrays can be made of any molecules whose synthesis involves sequential addition of units. This includes polymers composed of a series of attached units and molecules bearing a common skeleton to which various functional groups are added. Such polymers include, for example, both linear and cyclic polymers of nucleic acids, polysaccharides, phospholipids, and peptides having either α-, β-, or ω-amino acids, heteropolymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which will be apparent to anyone skilled in the art. Molecules bearing a common skeleton include benzodiazepines and other small molecules, such as described in U.S. Pat. No. 5,288,514, incorporated herein by reference in its entirety.

The present invention provides a novel method for non-destructive quality control method as applied to the synthesis of oligonucleotide arrays. One aspect of the invention relates to a non-destructive quality control methods for testing of a synthetic round of fabrication of a high density oligonucleotide array, wherein the synthetic round of fabrication comprises exposing a substrate in a flow cell, the substrate having a density exceeding 400 protected reactive species per cm², wherein the reactive species is protected with a photolabile protecting group and protected reactive species is selected from the group consisting of a protected terminal hydroxyl group of a linker that is bound to the substrate and directed away from the substrate, a protected 5′ hydroxyl group of a naturally or non-naturally occurring 2′-deoxyribonucleotide, and a protected 3′ hydroxyl group of a naturally or non-naturally occurring 2′-deoxyribonucleotide; selectively irradiating the substrate with light of a wavelength to remove a preselected amount of photolabile protecting groups to provide exposed reactive groups; providing an MOS block with a tube leading to the flow cell containing a first solution of a naturally or non-naturally occurring 2′-deoxyribonucleotide-3′-phosphoramidite, wherein the phosphoramidite is located at the 3′ position and the 5′ hydroxyl group is protected by a photolabile protecting group or the phosphoramidite is located at the 5′ position and the 3′ hydroxyl group is protected by a photolabile protecting group; performing an analytical measurement of the first solution to determine a stock solution concentration of phosphoramidite; flushing the flow cell with the first solution from the MOS block to couple the deoxyribonucleotides to the reactive groups, producing a waste phosphoramidite solution; testing a small volume of the waste solution to determine a waste solution concentration of phosphoramidite; comparing the stock and waste concentrations of phosphoramidites to establish a QC parameter.

In certain embodiments, the protected reactive group is a protected terminal hydroxyl group of a linker that is bound to the substrate and directed away from the substrate. In certain such embodiments, the selective irradiation, flushing the flow cell with the first solution, testing the phosphoramidite waste solution concentration, and establishing a QC parameter is performed repeatedly, thereby making an array of oligonucleotides.

In certain embodiments, the protected reactive group is a protected 5′ hydroxyl group of a naturally or non-naturally occurring 2′-deoxynucleotide. In certain such embodiments, the 2′-deoxynucleotide is selected from the group consisting of G, A, T, and C.

In certain embodiments, the protected reactive group is a protected 3′ hydroxy group of a naturally or non-naturally occurring 2′-deoxyribonucleotide. In certain such embodiments, the 2′-deoxyribonucleotide is selected from the group consisting of G, A, T, and C.

In certain embodiments of the non-destructive quality control method, the phosphoramidite concentration may be determined by HPLC.

In certain embodiments, the first solution comprises a 2′-deoxyribonucleotide-phosphoramidite, wherein the phosphoramidite is located at the 3′-position and the 5′ hydroxyl group is protected by a photolabile protecting group.

In certain embodiments, the first solution comprises a 2′-deoxyribonucleotide-phosphoramidite, wherein the phosphoramidite is located at the 5′-position and the 3′ hydroxyl group is protected by a photolabile protecting group. In certain such embodiments, the 2′-deoxyribonucleotide is selected from the group consisting of G, A, T, and C.

Suitable photolabile protecting groups include, but are not limited to, ortho-nitro benzyl deriviatives, nitropiperonyl (such as α-methyl-2-nitropiperonyloxycarbonyl (MeNPOC), pyrenylmethoxycarbonyl, nitroveratryl (such as 6-nitroveratryloxycarbonyl (NVOC)), nitrobenzyl, dimethyl dimethoxybenzyl, 5-bromo-7-nitroindolinyl, o-hydroxy-α-methyl cinnamoyl, and 2-oxymethylene anthraquinones. In certain embodiments, the photolabile protecting group is selected from MeNPOC and NVOC.

All publications and patent documents cited in this application are incorporated by reference in their entirety for all purposes to the same extent as if each individual publication or patent document were so individually denoted.

We claim:

1. A non-destructive quality control method for testing of a synthetic round of fabrication of a high density oligonucleotide array, wherein the synthetic round of fabrication comprises
exposing a substrate in a flow cell, the substrate having a density exceeding 400 protected reactive species per cm², wherein the reactive species is protected with a photolabile protecting group and protected reactive species is selected from the group consisting of a protected terminal hydroxyl group of a linker that is bound to the substrate and directed away from the substrate, a protected 5′ hydroxyl group of a naturally or non-naturally occurring 2′-deoxyribonucleotide, and a protected 3′ hydroxyl group of a naturally or non-naturally occurring 2′-deoxyribonucleotide;
selectively irradiating the substrate with light of a wavelength to remove a preselected amount of photolabile protecting groups to provide exposed reactive groups;
providing an MOS block with a tube leading to the flow cell containing a first solution of a naturally or non-naturally occurring 2′-deoxyribonucleotide-3′-phosphoramidite, wherein the phosphoramidite is located at the 3′ position and the 5′ hydroxyl group is protected by a photolabile protecting group or the phosphoramidite is located at the 5′ position and the 3′ hydroxyl group is protected by a photolabile protecting group;
performing an analytical measurement of the first solution to determine a stock solution concentration of phosphoramidite;
flushing the flow cell with the first solution from the MOS block to couple the deoxyribonucleotides to the reactive groups, producing a waste phosphoramidite solution;
testing a small volume of the waste solution to determine a waste solution concentration of phosphoramidite;
comparing the stock and waste concentrations of phosphoramidites to establish a QC parameter.

2. A method according to claim 1, wherein the protected reactive group is the protected terminal hydroxyl group of a linker.

3. A method according to claim 2, wherein the selective irradiation, flushing the flow cell with the first solution, testing the phosphoramidite waste solution concentration, and establishing a QC parameter is performed repeatedly, thereby making an array of oligonucleotides.

4. A method according to claim 1, wherein the phosphoramidite concentration is determined by HPLC.

5. A method according to claim 1, wherein the species is a 5′ hydroxyl group of a naturally or non-naturally occurring 2′-deoxyribonucleotide.

6. A method according to claim 5 wherein the 2′-deoxyribonucleotide is selected from the group consisting of G, A, T, and C.

7. A method according to claim 1, wherein the species is a 3′ hydroxyl group of a naturally or non-naturally occurring 2′-deoxyribonucleotide.

8. A method according to claim 7, wherein the 2′-deoxyribonucleotide is selected from the group consisting of G, A, T and C.

9. A method according to claim 1, wherein the first solution comprises a 2′-deoxyribonucleotide-phosphoramidite, wherein the phosphoramidite is located at the 3′ functionality and the 5′ hydroxyl group is protected by a photolabile protecting group.

10. A method according to claim 1, wherein the first solution comprises a 2′-deoxyribonucleotide-phosphoramidite, wherein the phosphoramidite is located at the 5′ functionality and the 3′ hydroxyl group is protected by a photolabile protecting group.

11. A method according to claim 10, wherein the 2′-deoxyribonucleotide is selected from the group consisting of G, A, T and C.
